(19) United States
(12) Patent Application Publication
(10) Pub. No.: US 2003/0170733 A1
(43) Pub. Date: Sep. 11, 2003

(54) Device and method for multiparallel synthesis and screening

(75) Inventors: Sergey A. Kozmin, Chicago, IL (US); Ying Wang, Chicago, IL (US)

Correspondence Address: K. Shannon Mrksich, BRINKS HOFER GILSON & LIONE, P.O. BOX 10395, CHICAGO, IL, 60610, US

(73) Assignee: University of Chicago.

(21) Appl. No.: 10/358,074

(22) Filed: Feb. 3, 2003

Related U.S. Application Data
(63) Continuation-in-part of application No. 10/172,337, filed on Jun. 13, 2002.
(60) Provisional application No. 60/361,827, filed on Mar. 4, 2002.

(51) Int. Cl. G01N 33/53; C12P 21/06; B01J 8/00; C12M 1/34

(52) U.S. Cl. 435/7.1; 435/68.1; 435/287.1; 422/187

(57) ABSTRACT

There is disclosed a device for multiparallel synthesis or screening of a chemical library that includes at least one, and preferably more than one, plate with a two-dimensional or three-dimensional array of wells. Openings are provided in the side walls between at least some of the wells, through which the fluid can flow between adjacent wells. At least one inlet for each plate is provided whereby fluids can be introduced into the array of wells in each plate and flow between adjacent wells in each of the plates through the openings in the side walls.

This application is a continuation of U.S. Nonprovisional Application Ser. No. 10/172,337 filed on Jun. 13, 2002, which claims the benefit of U.S. Provisional Application Serial No. 60/361,827 filed on Mar. 4, 2002, both applications being incorporated herein by reference.

### FIELD OF THE INVENTION

The invention generally relates to a device and method for a multiparallel synthesis and screening. In particular, the device and method of the invention relates to multiparallel synthesis and screening using arrays that allow multidimensional delivery of reagents along various axes or planes of the array.

### BACKGROUND OF THE INVENTION

The ability to rapidly generate and screen new collections of small molecules is crucial to the efficiency of the development of new therapeutic compounds. Over the past decade, combinatorial chemistry has emerged as an alternative approach for new lead discovery and optimization. Initially, the focus was on the synthesis of peptide libraries because of the readily availability of amino acids and well known coupling methods. Some of these methods are the split-pool technique, pin technology, iterative deconvolution, physical isolation of active beads using the Selectide method, and encoded libraries.

Recently, attention has focused on the development of libraries of small organic molecules, due to their superior oral bioavailability, cell wall penetration, and duration of action. While progress has been made, many challenges remain. The complexity and diversity of many existing small molecule libraries are based on the attachment of multiple groups to a common scaffold resulting in moderate perturbations of the same structure. While such libraries are effective in optimizing the initial lead structure, greater diversity is needed to discover new bioactive compounds. In addition, most of the existing libraries are generated by a split-and-pool method. In the split and pool technique, individual compounds are combined in one vessel for washing and deprotection and then divided again into separate portions for the next coupling. In the case where polymers are produced in the split-and-pool technique, a statistical distribution of sequences is obtained. Although efficient, this method has several limitations including the requirement for additional encoding, generation of small quantities of final products, and inability to conduct multicomponent condensations.

A system and method for a parallel synthesis of a library of compounds comprising a plurality of plates having wells that may be stacked to form a three-dimensional array has been described. U.S. Pat. No. 6,168,914 B1 describes a variation that comprises several well plates separated by one or more gaskets and stacked to form a three dimensional array having inlets for reagent delivery perpendicular to the plane of the array plates.

### SUMMARY

In accordance with one aspect of the invention, a device is provided for multiparallel synthesis or screening of a chemical library. The device includes a plate with a two-dimensional array of wells for receiving fluids. Each well has a bottom and side walls. Openings are provided in the side walls between at least some of the wells. Through these openings, fluid can flow between adjacent wells. At least one inlet is provided for a plate whereby fluids can be introduced into the array of wells and flow between adjacent wells through the openings in the side walls.

Preferably, the device of this first aspect includes more than one plate joined together to thereby form a three-dimensional array of wells. In addition, the wells in each plate are preferably arranged in similar rows and columns. Also, there is preferably an inlet associated with each row and column and a controllable fluid supply associated with each inlet.

In accordance with a second aspect of the invention, the device includes an array comprising rows of wells for receiving fluids, with each well comprising a bottom and side walls. Openings in the side walls are aligned along each row. The device also includes at least one elongated element which is inserted through at least some of the openings along at least one row. The flow of fluids along said at least one row is controlled by the elongated element reducing or closing the openings it passes through. In one embodiment of this second aspect, the elongated element is operated by selectively being inserted into and removed from the row of wells. In another embodiment, the elongated element is operated by changing the extent of insertion less than the length of a single well. In this embodiment, the elongated member is preferably a tapered pin and the openings are progressively smaller along the row toward the end of the row. In yet another embodiment of the second aspect, the elongated element is operated by rotating the element about its longitudinal axis.

In a third aspect, a device according to the present invention comprises wells for receiving fluids arranged in rows and columns. Each well comprises a bottom and side walls. The device includes a first set of openings in the side walls aligned along each row and a second set of openings in the side walls. The openings in the second set of openings are aligned along each column. The device also includes a first set of elongated elements, each adapted to be inserted through the first set of openings in a single row. In addition, the device includes a second set of elongated elements, each adapted to be inserted through the second set of openings in a single column. The flow of fluids along each row is controlled by the elongated element of the first set passing therethrough by reducing or closing the openings therein. Also, the flow of fluids along each column is controlled by the elongated element of the second set passing therethrough by reducing or closing the openings therein.

In accordance with a fourth aspect of the invention, a device for multiparallel synthesis or screening is provided that includes a three-dimensional array formed from at least two plates comprising a plurality of wells for receiving a fluid. The device includes means for introducing the fluid into a plurality of inlets at two or more adjacent surfaces of the three-dimensional array. The fluid flows from a first of the plurality of wells to one or more other wells selected from the plurality of wells through openings in the plurality of wells. A bottom portion of each of the plurality of wells is sealed to prevent the flow of the fluid from a well of a first plate of the at least two plates to a well of a second plate.

In a fifth aspect of the invention, a device for multiparallel synthesis or screening is provided that comprises an array formed from at least two plates comprising a plurality of wells for receiving a fluid. A means for allowing a flow of the fluid between at least two of the plurality of wells in a substantially parallel direction relative to a plane of one of the at least two plates is also provided. The fluid is introduced through an opening on one side of the array. In addition, the fluid flows from a first of the plurality of wells to one or more other wells selected from the plurality of wells through openings in the plurality of wells.

According to a sixth aspect of the invention, a device for multiparallel synthesis or screening includes an array formed from at least two plates comprising a plurality of wells for receiving a fluid. The device of this sixth aspect includes a means for introducing a fluid into at least three sets of wells selected from the plurality of wells. An opening on one side of the array introduces the fluid. In the sixth aspect, the fluid flows from a first of the plurality of wells to one or more other wells selected from the plurality of wells through openings in the plurality of wells. Each set of wells is oriented perpendicularly relative to two of the three sets of wells.

In a seventh aspect of the invention, a device for multiparallel synthesis or screening is provided that comprises an array formed from at least two plates comprising a plurality of wells for receiving a fluid. The device also includes means for introducing the fluid into one or more of the plurality of wells. In addition, the device includes a plurality of removable sealing elements each of which extends between at least two openings in at least one of the plurality of wells when in a sealing position.

Preferably, in the device of the seven aspect, at least one of the plurality of removable sealing elements is selected from a group consisting of a rod-shaped sealing element, tapered sealing element, and a cylindrical sealing element. The sealing elements are preferably in the form of stainless steel tapered pins. Preferably, the fluid is introduced through one or more openings in at least one of the plurality of sealing elements.

In an eighth aspect of the invention, a device is provided that comprises an array comprising wells for receiving fluids arranged in rows and columns, each well comprising a bottom and side walls. The device of this eighth aspect includes a first set of openings in the side walls, the openings in the first set of openings being aligned along each row. The device also includes a second set of openings in the side walls, the openings in the second set of openings being aligned along each column. In addition, the device includes a first set of elongated elements, each adapted to be inserted through the first set of openings in a single row. A second set of elongated elements, each adapted to be inserted through the second set of openings in a single column is also included in the device. In this eighth aspect, the flow of fluids along each row is controlled by the elongated element of the first set passing therethrough, reducing or closing the openings therein. Further, the flow of fluids along each column is controlled by the elongated element of the second set passing therethrough, reducing or closing the openings therein.

In accordance with a ninth aspect of the present invention, a device for multiparallel synthesis or screening of a chemical library is provided that includes a three-dimensional array formed from at least two plates joined together. Each plate includes a two-dimensional array of wells for receiving fluids and/or solid materials. The wells in each plate are formed into rows and columns. Each plate includes a bottom and four side walls. The device of the ninth aspect includes openings in all four of the side walls of all of the wells in the arrays, except the wells at the end of each row or column. A first set of the openings allows fluids to flow between adjacent wells along each row, while a second set of openings allows fluids to flow between adjacent wells along each column. The device of the ninth aspect also includes a first set of inlets for adding fluids into each row and a second set of inlets for introduction of fluids into each column. The first set of openings is adjacent the bottom of the wells and the second set of openings is adjacent the upper portion of the wells. The level of fluid in the wells is maintained below the second set of openings when the device is in a first orientation with the bottoms of the wells below the upper portions of the wells. The level of fluid in the wells is maintained below the first set of openings when the device is inverted to a second orientation with the upper portions of the wells below the bottoms of the wells. Thus, selective fluid communication occurs between the wells in the rows but not between the wells in columns when the device is in its first orientation. Fluid communication occurs between the wells in the columns but not between the wells in the rows when the device is inverted to its second orientation.

The device of the ninth aspect preferably includes a solid reaction support in at least some of the wells. The device may also include a fluid introduction conduit associated with at least some of the inlets. Preferably, the diameter of the fluid introduction conduit is smaller than the diameter of the openings. The fluid introduction conduit preferably passes through the inlet and through all of the first set of openings in a row whereby fluid is introduced into the last well in the row first and flows into the remaining wells through said openings. Preferably, the conduit is selectively inserted into and retracted from the row.

In a tenth aspect of the invention, a method for synthesizing or analyzing compounds is provided that includes introducing a first fluid into a plurality of wells of an array formed from at least two plates through at least one opening in each wall of the plurality of wells. The method of the tenth aspect also includes introducing a second fluid into the plurality of wells through the at least one opening in each wall of the plurality of wells. The first and second fluid is introduced through an opening on one side of the array. In addition, the first and second fluid flow from a first well to one or more other wells in a substantially parallel direction relative to a plane of one of the at least two plates.

Preferably, the method of the tenth aspect further includes inserting at least one elongated element into at least one opening in one or more wells. At least one elongated element preferably extends between at least two openings in at least one of the wells when in a sealing position. Preferably, the method of the tenth aspect also includes performing one or more washing steps after introducing one or more fluids into the wells. The fluid may be added using a syringe or tube. Preferably, between about 0.01 milligram to about 20 milligram of at least one member of a library of compounds is produced from a mixing of at least two fluids. The fluid can be added into the wells using a pump. Solid-phase supports that can be used in a synthesis or screening include materials such as polymers, resins, metals, glass beads, silica supports, gel or gel-type solid-phase supports, encapsulated gel solid-phase supports, macroporous supports, modified surfaces, and composite particles.” A functionalization, linking, or cleaving step is preferably performed in at least one set of wells. The fluid may include small molecule reactants. Alternatively, the fluid may include molecules with dendritic structure.

In an eleventh aspect of the invention, a method for synthesizing or analyzing compounds is provided that includes introducing a fluid into a first set of wells of an array. A fluid is then introduced into a second set of wells that lie perpendicularly to the first set of wells of the array. Following the addition into the second set of wells, a fluid is added into a third set of wells that lie perpendicularly to each of the first set of wells and the second set of wells of the array. Preferably, in the method of the eleventh aspect, any of the first set of wells, second set of wells, and the third set of wells corresponds to a column of wells or a row of wells.

In a twelfth aspect of the invention, a method for synthesizing or analyzing compounds includes introducing a fluid into a first set of inlets at a first surface of a three-dimensional array that includes several wells. The method of the twelfth aspect also includes introducing a fluid into a second set of inlets at a second surface of the three-dimensional array. In this method, the first surface of the three-dimensional array lies adjacent to the second surface of the three-dimensional array.

In a thirteenth aspect, a method is provided for synthesizing or analyzing compounds comprising introducing a fluid through a first set of openings in a first set of side walls in an array. The array is then rotated through a certain angle. Following the array rotation, a fluid is then added through a second set of openings in a second set of side walls in the array.

The present invention includes various non-limiting embodiments or modifications of the invention, those embodiments and modifications including combinations of one or more features of two or more aspects of the invention described above. Several embodiments and modifications of the invention are discussed in details below.

### BRIEF DESCRIPTION OF THE DRAWINGS

FIG. 1 is a schematic representation of a matrix array of molecular libraries.

FIG. 2 is a perspective view of a well of the device and a 2×2×2 and 5×5×5 arrays for use in multiparallel synthesis.

FIG. 3 is different perspective views of a 125 compound 3-dimensional multiparallel synthesizer.

FIG. 4 is a perspective view of a 1000-compound array according to the present invention formed by stacking several individual plates.

FIG. 5 is a perspective view of a 1000-compound array and several of the possible assemblies using the array.

FIG. 6 is a perspective view of a conventional plate and a three-dimensional array according to the present invention.

FIG. 7 is a cross-sectional view of a solid-phase support dispenser and an array plate illustrating a method for dispensing the beads into the array wells.

FIG. 8 is a cross-sectional view of three wells with different sizes each containing either a solid-phase support or dendrimers.

FIG. 9 is a perspective of a three-dimensional array illustrating a multidimensional array operation according to the present invention.

FIG. 10 is a cross-sectional view of a portion of an array plate illustrating a method according to the invention for adding and withdrawing fluid from an array containing solid-phase support.

FIG. 11 is a top view of a unit layer or plate comprising an array of wells, each of which contains a solid-phase support, and a three-dimensional multiparallel synthesizer with connections to reagent reservoirs.

FIG. 12 is a perspective view of a preferred format for a 96-well plate according to the present invention.

FIG. 13 is a perspective view of a preferred format for a 384-well plate according to the present invention.

FIG. 14 is a perspective view of a three-dimensional array according to the present invention that can be used in the synthesis of a 125-member library of tripeptides.

### DETAILED DESCRIPTION OF THE INVENTION

The invention is directed to various devices and methods for multiparallel synthesis and screening of chemical libraries. As used herein, the term “screening” refers to any method or combination of methods of chemical or biochemical analysis. Such libraries can comprise compounds, such as small molecules and biomolecules such as peptides.

These methods and devices are based on multi-dimensional arrays of interconnected wells. As used herein, an “array” refers to a plurality of wells. Each well includes at least a bottom and side walls. The bottom is preferably flat and horizontal. Alternatively, the well may be formed in a shape, such as hemispherical, wherein the bottom is curved. Each well preferably also includes a top. Preferably, the top is formed by stacking a plate on top of another.

As used herein, the “side wall” of a well refers to a side or surface of a well that defines its boundary (or volume). The sides of a wall of a given well may assume any shape. For example, a semi-spherical well can have at least a portion of the well wall that is curved and continuous while a square or rectangular well will have four, generally flat, walls corresponding to its four vertical sides that are perpendicular to the bottom side or bottom wall of the square or rectangular well. As used herein, a “side wall” of a well refers to a vertical side of a well that is oriented more or less perpendicularly to a bottom side or wall of a well. In the present invention, an opening in a wall of a well is generally and preferrably located in a side wall of a well.

Preferably, the wells have a rectangular footprint with four side walls. Most preferably, the wells have a square footprint with four equal-sized side walls. Alternative designs for wells are also possible. For example, the wells may have a cylindrical shape. This embodiment with cylindrical wells could be described as having a single, curved side wall. However, for convenience, this embodiment will be described as having four side walls, each corresponding to one fourth of the cylindrical wall. In addition, the term bottom is used

These wells are formed within plates. Preferably, the wells are arranged in rows and columns on each plate to thereby define a two-dimensional array. More preferably, multiple plates are joined together to produce three-dimensional arrays. Most preferably, like plates are stacked on top of each other to thereby produce a three-dimensional array with horizontal and vertical rows and columns of wells.

Preferably, each row of each plate includes an inlet whereby fluids are passed into that row. More preferably, each column also includes an inlet.

Preferably, a fluid supply is associated with each inlet. The fluid supply is preferably in the form of a tube or conduit, such as the tubes 120,122 in FIG. 11B. Alternatively, fluid is supplied to multiple inlets by a single fluid supply. For example, one or more robotically controlled pipettors can be used to deliver fluid to multiple inlets.

Preferably, there is a fluid supply controller which controls the fluid going through the fluid supply into the inlets. As used herein, a “fluid controller” or “fluid supply controller” refers to a device, set-up, or mechanism that permits a selective transfer or addition of one or more fluids from at least one fluid source to one or more fluid destinations. A fluid controller or fluid supply controller can also control one or more parameters involving a fluid such as its flow rate, temperature, or rate of mixing with another fluid. Preferably, the fluid supply controller includes various valves and pumps, all actuated by a programmable microprocessor.

A fluid source or a fluid destination includes, but is not limited to, a well, plate, array, tray, flask, beaker, and other types of containers, whether conventional, homemade, or custom-built.

The devices and methods of the present invention provide several useful features. For example, the three-dimenional arrays allow diversification of the library through reagent introduction or functionalization along two or three perpendicular directions. Compounds can be cleaved without removing the beads from an array. From 0.01-20 mg of individual library members may be produced and multiparallel synthesis of greater than 1,000 individual compounds may be obtained. The devices and methods of the invention also provide improved efficiency compared to that of traditional plates using, for example, a 384-well plate. In addition, the devices and methods of the invention allow both solid-phase and solution-phase synthesis.

The invention also can allow for positional or spatial encoding. Thus, there may be no need for additional complicated and time-consuming steps to identify the structure of library members, such as electronic encoding, graphical encoding, chemical encoding, spectrometric encoding, or deconvolution techniques. Preferably, the compounds can be identified simply from their particular positions in the array.

As noted above, a three-dimensional array of the invention is preferably formed by stacking two or more layers or plates of wells to form a three-dimensional matrix. This is illustrated in, for example, FIG. 4. FIG. 4 shows several plates 40 comprising wells 41 that are stacked to form a three-dimensional array 45. In FIG. 4, the plates 40 that are stacked to form the array 45 are shown with openings 42 on two adjacent sides of the plates 40 through which elongated elements may be inserted. The array plates may be held together by a top and bottom layers or plates that are connected using, for example, screws to clamp tight the middle plates, such that the fluids or solid material in the wells in each plate are prevented from leaking. If desired, one or more layers of materials that are nonreactive or resistant to the reagents or solvents may be placed between the plates to further enhance the sealing between the layers of wells.

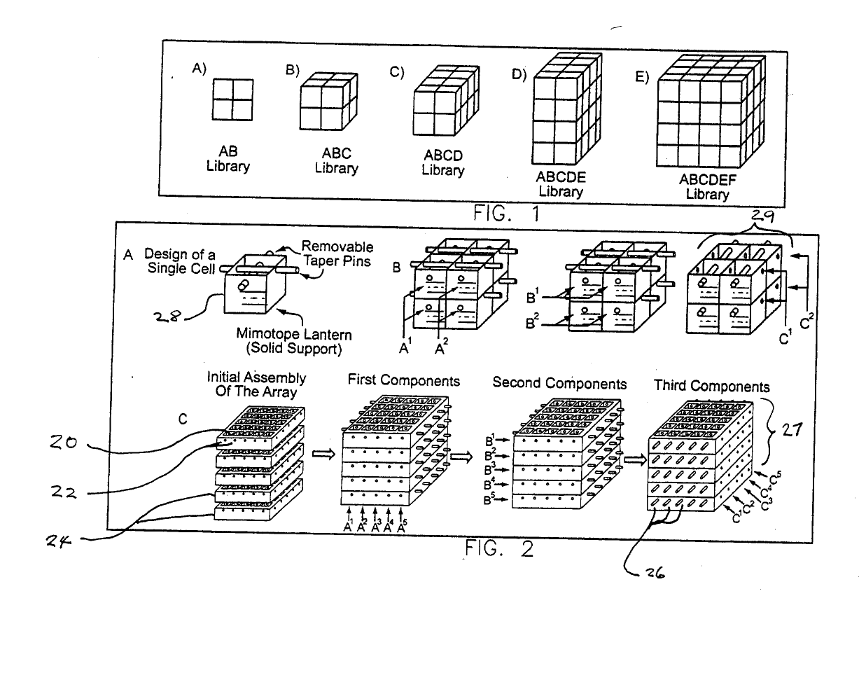

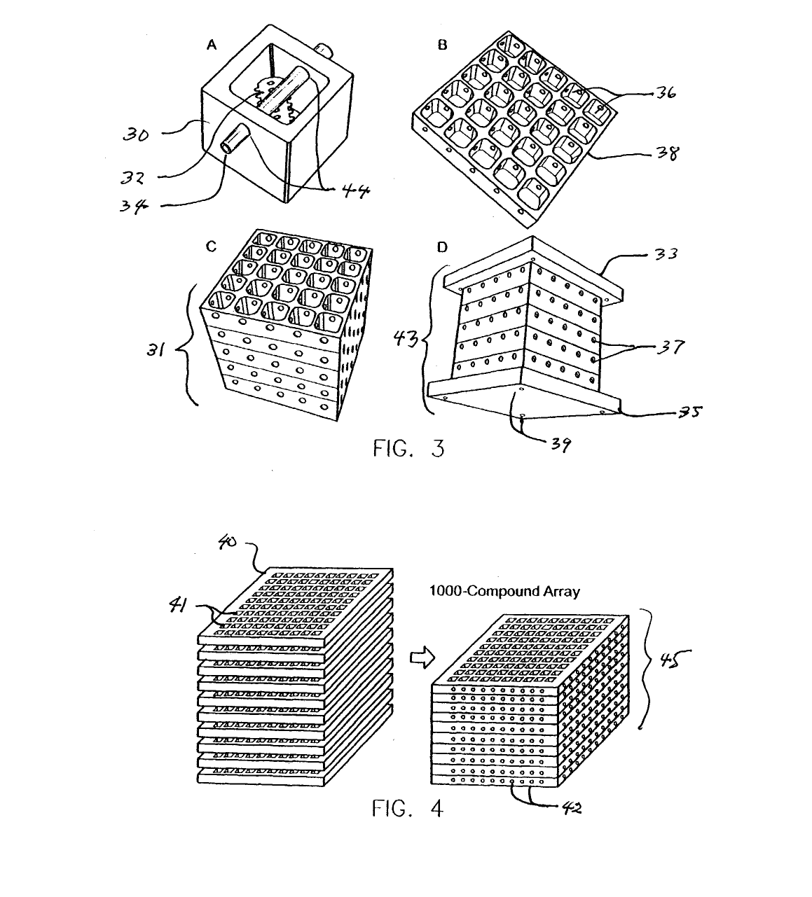

FIG. 1 shows a schematic representation of molecular libraries in the form of a single cubical array. FIG. 2 shows an exemplary well 28 and a 2×2×2 and 5×5×5 arrays 27, 29 for use in multiparallel synthesis. FIG. 2 shows plates 24 comprising wells 20 and openings 22. FIG. 2 also shows plates 24 of a 5×5×5 array 27 stacked to form a three-dimensional array 27, in which the plates 24 have elongated elements 26 going through from one side of a plate 24 to the opposite side of the plate 24 such that the elongated elements 26 can be manipulated on either side. FIG. 3 shows a 125-well three-dimensional multiparallel synthesizer 43 comprising a top clamping plate 33 and a bottom clamping plate 35 that are used to clamp tight the plates 38 in-between the top clamping plate 33 and bottom clamping plate 35, in which the plates 38 are stacked to form a three-dimensional array 31. Screws 39 can be used to clamp the plates using the clamping plates 33, 35. In the array shown in FIG. 3, each plate has openings 37 on all sides of the plates 38. FIG. 3 also shows a diagram of a well 30 that contains a solid support 32 and an elongated element 34 that goes through the well through openings 44. Preferably, the different configurations of the arrays are modifiable into, or compatible or usable with, 384-well plates, which are commercially available.

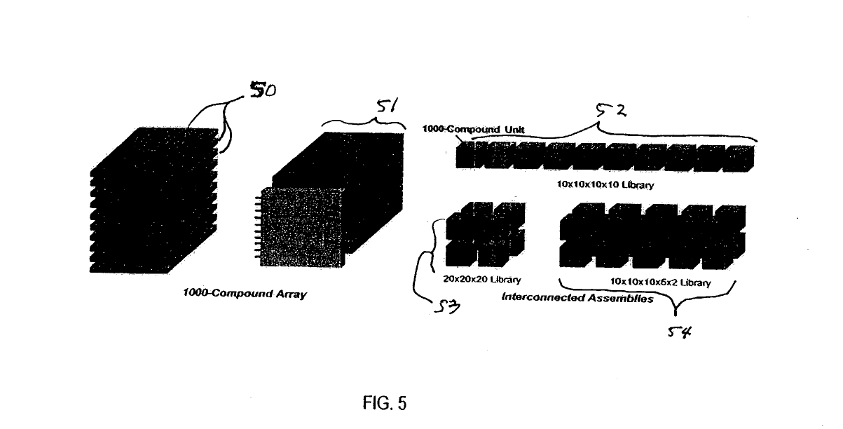

FIG. 5 shows a 1000-compound array 51 formed by stacking several plates 50. This array 51 can be used for generating a variety of small-molecule libraries. The array 51, which is reusable, can be prepared from polypropylene or teflon. An attachment unit allows the addition of ten reaction components to ten layers of the cube simultaneously. The entire process of generating a 1000-compound library can be performed in three steps. A combination of several 1000-compound arrays allows the preparation of 8,000 or more compound libraries. FIG. 5 shows various possible array configurations 52, 53, 54 such as a 20×20×20, 10×10×10×10, and a 10×10×10×5×2 arrays.

The arrays of the invention can be used alone or can be interconnected to additional arrays. Such connections may be driven by one or more pumps. Thus, the products or intermediates produced in a given array may be directed to another array which is directly or indirectly connected. When described as being “in fluid communication” with each other, these two or more arrays are connected in some fashion using connecters (e.g., tubes) through which fluid can flow, whether or not fluid between the at least two arrays is static or flowing, and whether or not the fluid flows continuously or discontinuously. The intervening arrays may be used for the washing steps or they may be used to introduce one or more reagents through one of the possible points or sides of entry. A functionalization, linking, or cleaving step can be performed in one or more of the arrays. The transfer or addition of reagents can be conveniently accomplished with the use of a fluid controller or fluid supply controller as described above.

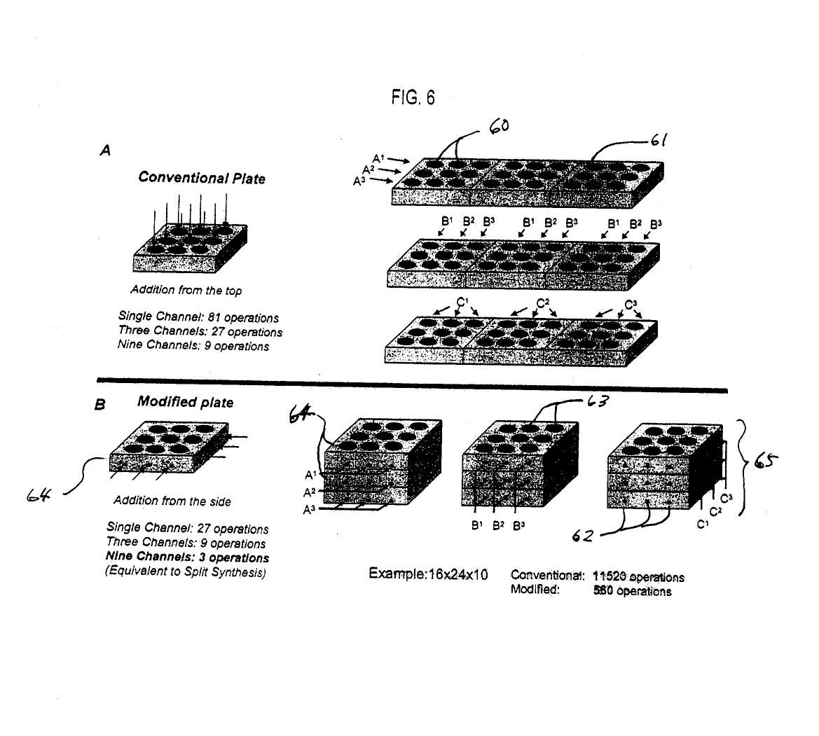

As shown in FIG. 6A-B, an array according to a preferred embodiment of the present invention provides higher efficiency and throughput compared to those of the conventional approach. FIG. 6B shows an array 65 formed by stacking several plates 64 comprising wells 63 that have openings 62 on at least one side of the plates 64. As shown in FIG. 6B, addition of the required reagents according to the present invention only involves 27 operations using a single-output delivery means (e.g., a pipette), 9 operations with three-output delivery means, and 3 operations with a 9-output delivery means, not including the washing steps that may be used between chemical steps. In contrast, a conventional plate 61 having a plurality of wells 60 shown in FIG. 6A would require 81, 27, and 9 operations, respectively. For a synthesis of a 6×24×10 ABC library using a stack of ten 384-well plates, the present invention would involve only 560 operations while conventional arraying would require 11520 operations.

The wells of the array receive and hold reagents, solvents, and/or one or more products produced in one or more reactions that occur in the wells. As used herein, the terms “reagent” or “fluid” include substances, reactants, reaction products, solvents, solutions, dispersions, emulsions, compounds, and mixtures, any one of which may be in a solid, liquid, or gaseous phase. Preferably, the wells are made of materials such as stainless steel, aluminum, polypropylene, polyethylene, or PTFE, other suitable materials, as well as combinations of materials. The materials of which the wells are made of are preferably resistant or nonreactive to the reagents or solvents used in the synthesis.

The wells comprising the array may number from less than 100, but they may also range from 1,000 wells or more per array. Preferably, the smallest side of a well is at least from about 1.5 mm to about 5 mm, more preferably from about 2.5 mm to about 4 mm, most preferably from about 3.4 mm to about 3.8 mm.

Preferably, an arrayer or dispenser is used to load the wells with solid-phase support. The solid-phase support dispenser can be made of a plate with wells, wherein each well has a desired volume and a position that substantially matches or coincides with the position of a well in an array plate. The diameters or the longest cross-sectional length of the dispenser wells are preferably smaller or substantially the same as those of the array wells. The dispenser can be made of materials such as aluminum, stainless steel, polypropylene, or polyethylene.

The solid-phase support particles or pieces can be uniformly distributed over a dispenser by placing a certain amount of solid-phase support on the dispenser plate and spreading the solid-phase support throughout the plate using a relatively rigid material such as a ruler or a solid plate with a flat or straight side or edge.

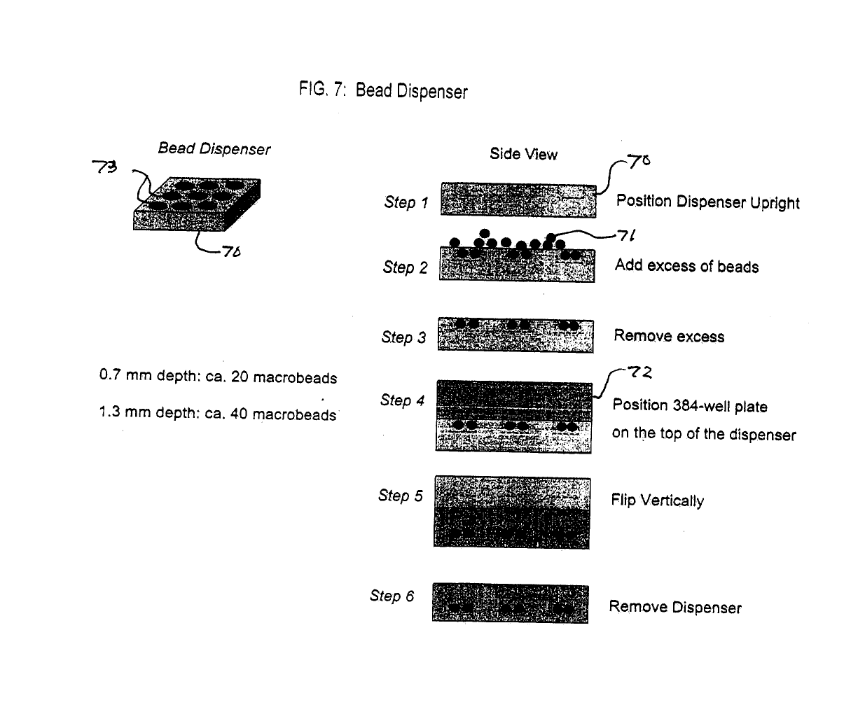

FIG. 7 illustrates how the array wells can be filled using a dispenser or arrayer 70. The dispenser wells 73 can be filled with solid-phase support particles or pieces 71 with one or more wiping motions of a rigid material with a flat or straight edge. During the wiping motions, the straight edge or surface of the rigid material is preferably kept flat against the surface of the dispenser 70 so that excess solid-phase support 71 can be removed from the dispenser 70. A tool or material such as a brush or a piece of plastic or wood may be used to evenly distribute the solid-phase support particles or pieces 71 over the dispenser wells 73. After filling the dispenser wells 73 with the desired amount of solid-phase support 71, an array plate 72 can then be placed on top of the dispenser 70, with its well openings facing the openings of the dispenser wells 73 so that the wells in both plates substantially coincide. Flipping the two plates such that the array plate 72 sits below the dispenser 70, and shaking or tapping the plates if necessary, would substantially transfer the solid-phase support 71 from the dispenser 70 to the array plate 72. Other methods of dispensing or adding solid-phase support into the array 72 may also be used.

In one preferred configuration of the dispenser, the depth of the dispenser wells is similar or substantially the same as the diameter of the solid-phase support particles so that a monolayer of the solid-phase support particles are formed on each well of the dispenser. Different number of solid-phase support layers in the array wells may be obtained by using array plates with wells having a desired depth. If desired, different sections of a dispenser or array plate can have different well depths or volumes so that the amount of solid-phase support in the wells can be varied for a given dispenser or array plate.

Solid-phase supports that can be used in a synthesis or screening include materials such as polymers, resins, metals, glass beads, silica supports, gel or gel-type solid-phase supports (e.g., gel-type polystyrene resin solid-phase support, copolymer solid-phase supports (e.g., poly(styrene-oxyethylene graft copolymer supports), encapsulated gel solid-phase supports, macroporous supports, modified surfaces, and composite particles. A polymer that partly or substantially makes up a solid-phase support preferably includes polypropylene, polystyrene, chloroacetyl polystyrene, carboxypolystyrene, polystyrene-CHO, chloromethylated polystyrene polyamide, or polystyrene-poly(ethylene glycol) graft. Resins such as those made of imidazole carbonate resin, polyacrylamide resin, benzhydrol resin, p-nitrophenyl carbonate resin, diphenylmethanol resin, trityl alcohol resin, hydroxymethyl resin, or triphenyl methanol polystyrene resin, or their various combinations may also be used. Solid-phase supports such as resin beads and lanterns sold under the name SynPhase™ lanterns are commercially available.

Preferably, the dimension of the solid-phase support is no less than between about 0.6 mm to 0.7 mm, more preferably no less than about 0.5 mm. Polystyrene beads with dimensions ranging from approximately 500-600 μm are commercially available. The amount of solid-phase support placed in the wells of an array depends on factors such as the desired amount of products to be synthesized, as well as the well volume and the extent of swelling of the solid-phase support upon contact with a solvent or reagent. For example, a well may include between about 1-20 beads. Preferably, the synthesis produces at least about 1 mg of a reaction product, more preferably at least about 3-4 mg of a reaction product.

The type of solid-phase support to be used partly depends on one or more factors such as the desired amount of material to be synthesized (the loading capacity), compatibility of the chemistry intended for the library synthesis, and mode of attachment and cleavage of materials from the solid-phase support.

Preferably, a solid-phase support is prefunctionalized and may contain one or more functionalities or linkers. A solid-phase support may be functionalized with one or more chemically reactive groups that are used to attach a linker to a solid-phase support. Examples of these chemically reactive groups include, but are not limited to, isocyanates, carboxylic acids, esters, amides, alcohols, isothiocyanate, amines, and halomethyl groups. If desired, a solid-phase support that does not contain any functionalities or linkers may be used. Various non-functionalized solid-phase supports are commercially available such as certain types of SynPhase™ Lanterns.

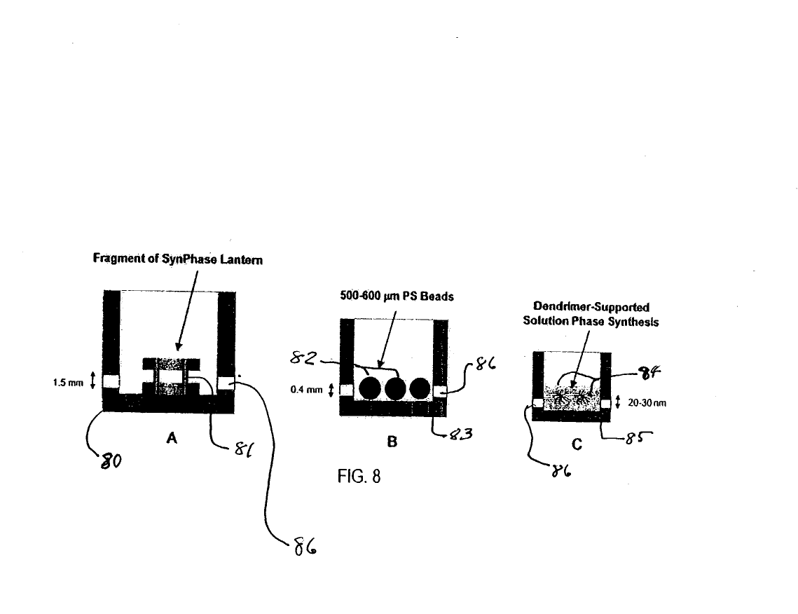

An array according to the present invention can be used in a synthesis or screening not only through the use of conventional solid-phase supports but also through the use of dendrimers or dendrimer-like molecules. A dendrimer-supported synthesis allows further miniaturization and offers the advantages of a solution-phase synthesis. As used herein, the term “dendrimer” includes dendrimers (as those molecules are known in the art) and molecules with dendritic or dendrimer-like structure. As used herein, a dendritic structure refers to a dendrimer structure or a structure similar to that of a dendrimer. Preferably, the dendrimer-supported synthesis is used in combination with nanoporous membranes. FIG. 8 shows wells 80, 83, 85 containing either dendrimers 84 or a solid-phase support 81, 82. As shown in FIG. 8, suitable diameters of well openings 86 are about 1.5 mm, about 0.4 mm, and between about 20-30 nm for a SynPhase lantern fragment, polystyrene bead, and dendrimer-supported synthesis, respectively.

Preferably, a multidimensional array operation, such as reagent addition or removal, is achieved mainly through the use of an opening between at least two wells. Thus, for example, an array operation is performed without the use of a material that either partially obstructs or substantially seals an opening between wells.

In a preferred embodiment of the ninth aspect, a first set of openings is positioned in a lower portion of the well of an array, i.e. adjacent the bottom, while a second set of openings, which is oriented perpendicularly to the first set, is situated in an upper portion of the well, i.e. adjacent to the top. As used herein in the term “adjacent to the bottom” simply means that the opening is closer to the bottom than it is to the top when the array is in its starting orientation. Likewise, the term “adjacent to the top” simply means that the opening is closer to the top than it is to the bottom when the array is in its starting orientation.

In this embodiment, each opening in a given pair preferably lies on opposite sides or walls of a well. Addition of reagents is preferably carried out using only the lower openings, dispensing the volume of liquid necessary to cover a solid-phase support without reaching the upper openings of the well.

Addition of a reagent in a direction perpendicular to a previous addition of a reagent through one set of openings can be achieved by turning the array upside down such that the pair of openings previously in the upper portion of each well are positioned into the lower portion of the well, that is, the upper pair of openings becomes the lower pair of openings in the well after an operation such as a 180-degree flipping of the well. To prevent a solid-phase support from moving or flowing from one well to another, the dimensions of the solid-phase support should be such that it cannot go through an opening between wells. Preferably, the diameter of an opening between wells is narrower than the narrowest dimension of a solid-phase support inside the wells. More preferably, the dimension of the solid-phase support is no less than about 500 μm.

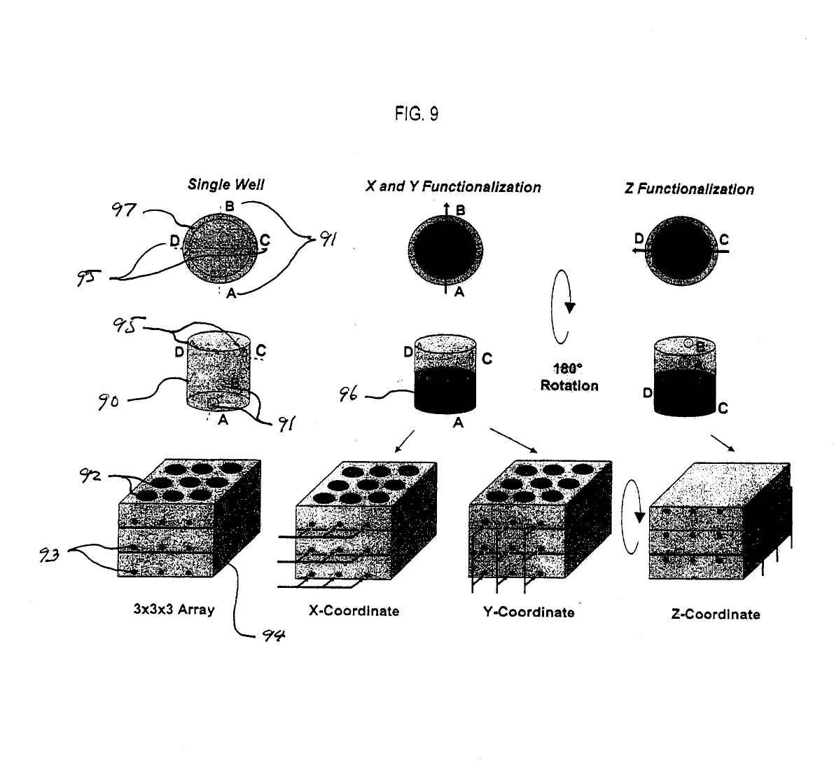

FIG. 9 illustrates the above array-flipping operation involving the addition of a reagent into a 3×3×3 array formed by stacking plates 94 that have wells 92, and openings 93 on at least one side of the plates. FIG. 9 depicts a pair of openings 91 (A and B) in a lower portion of a well 90, as well as a pair of openings 95 (D and C) that are located in an upper portion of the well 90 and that are oriented perpendicularly to openings 91. FIG. 9 also depicts a top view 97 of a well that includes openings 91, 95. FIG. 9 depicts, as described above, a 180-degree flipping or, in this case, a vertical inversion of an array to perform a crosswise or perpendicular addition of reagents 96 relative to a first reagent-addition step.

An array preferably has inlet ports for allowing or facilitating the addition of a reagent into at least one array well. A fluid supply, preferably a tube made of a relatively rigid, non-reactive material, can be used to bring fluid to the inlet port, by inserting or attaching it into an opening of a well on a surface of the array. If desired, fluid supplies can be made a more or less permanent part of an array. Thus, for example, one end of a tube can be inserted or connected into an inlet port of the array while the other end of the tube may be connected to another array or a reagent reservoir or container. As used herein, the term “fluid source” refers to any source of fluid or reagent, such as a fluid container or reservoir, including a plate or array that contains and supplies a fluid to wells of another array. A fluid source also includes any source of fluid or reagent that is connected between arrays. For example, a fluid source can be a fluid reservoir between arrays that may be opened or closed using, for example, valves to allow or block the flow of a fluid into an array on either side of the fluid reservoir.

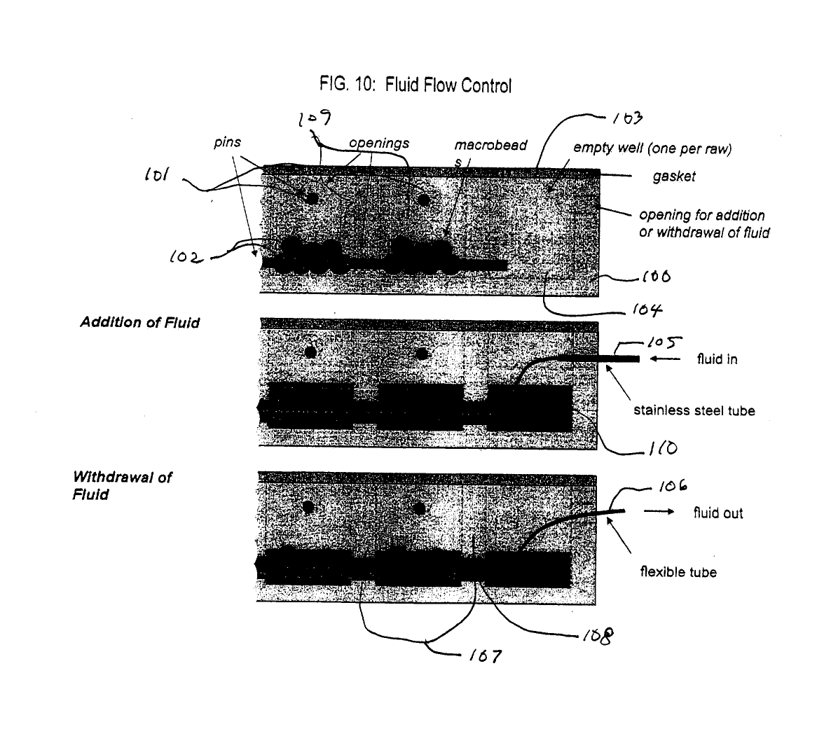

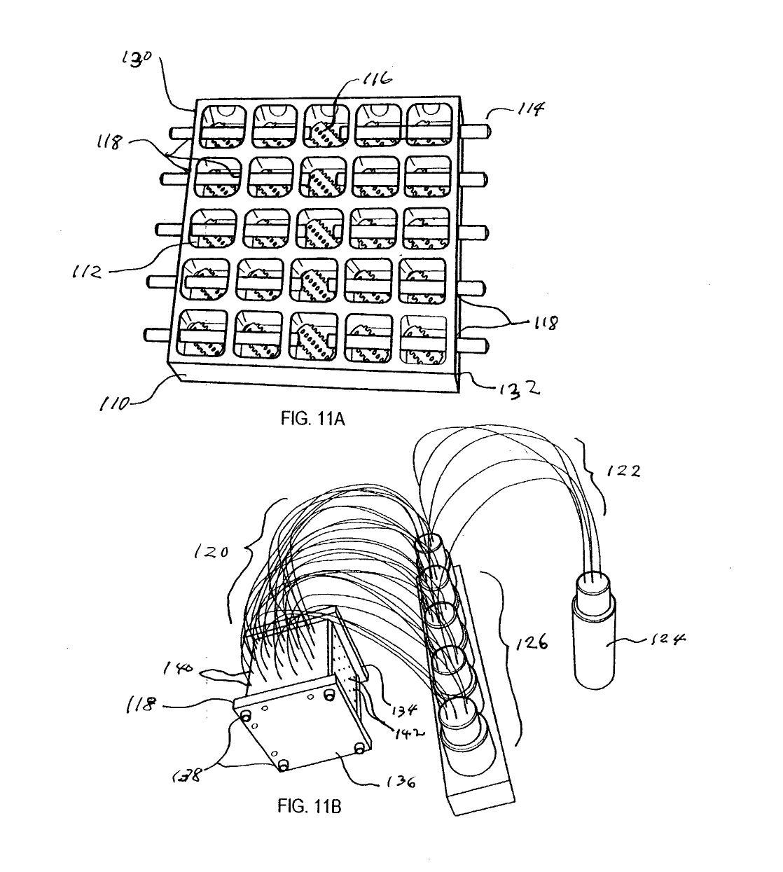

FIG. 10 illustrates array operations involving the addition into or withdrawal of a fluid 110 from array wells 109 of a plate 100, in which the array wells have top openings 101 and bottom openings 108 through which fluid 110 can flow. A top layer 103, such as a plate or a sealing film, covers the wells 109. In FIG. 10, at least one of the array wells 104 does not contain a solid-phase support 102. In FIG. 10, the wells 109 containing fluid 110 and solid support are shown to have openings 101, 108 through which pins may be inserted. Performing array operations using this particular array configuration can facilitate the addition or removal of a fluid 110 from array wells 109 that contain both fluid 110 and solid-phase support 102. To introduce a fluid 110 into the array wells 109, the fluid 110 is preferably added into the wells 109 through an inlet port 105 made of a material such as a stainless steel tube. A flexible tube connected to a fluid reservoir may be connected to the inlet port 105 when adding the fluid 110. To withdraw a portion or all of the fluid 110 in the array wells 109, a flexible tube 106 may be inserted through one of the openings 101, 108 of an outermost well. The fluid 110 may then be removed from the wells 109 using the tube 106 and a suction device such as a suction pump.

To reduce or eliminate problems relating to surface tension, the well openings can be partially obstructed using a non-reactive material. The nonreactive material can be an elongated element that spans at least two sides or openings of a well. Such elongated elements may be in the form of a solid or hollow pin, rod, or tube. Elongated elements can be used as a fluid introduction conduit, as a sealing element, or both. An example of a suitable elongated element is one that has an average diameter of about 0.7 mm if a well opening is about 1.2 mm. The elongated element may be configured such that it can be operated by rotating the element about its longitudinal axis. For example, an elongated element may include one or more semi-circular or fan-shaped sections perpendicular to the elongated element's longitudinal axis. The semi-circular sections are preferably located along the elongated element at distances that coincide with the distance between, for example, each pair of opposite side walls or each pair of openings in opposite side walls of a well. Preferably, the openings have a matching semi-circular sections that would allow the openings in the walls to be partially or completely sealed when the elongated element with semi-circular sections are rotated along the element's longitudinal axis.

A device of the invention preferably includes means for introducing two or more fluids or solids into three or more sets of wells, wherein at least three of the sets of wells are oriented perpendicularly to each other. A set of wells may correspond to either a column of wells or a row of wells. As used herein, a set of wells is “aligned” when the wells in that set are arranged in a more or less linear fashion. As used herein, the term “row” refers to a set of elements such as wells that are aligned, that is, arranged in a more or less linear fashion. A row of wells in the arrays of the invention are generally contiguous, i.e., two successive wells in a row of wells typically share a boundary such as a wall. In a two-dimensional array, such as one represented by a plate of wells, a “column” of wells would be defined as a more or less linear arrangement of wells, preferably contiguous, that is oriented perpendicularly relative to a row of wells. Thus, if a more or less linear arrangement of wells is defined or referred to as a row of wells, a perpendicular arrangement of a second, more or less linear, arrangement of wells would be defined or referred to as a column of wells. In a three-dimensional array, a column generally refers to a vertical or an upright arrangement of wells relative to a plane of an array plate. Thus, if a column in a three-dimensional array corresponds to the z-axis, a first row and a second row (or vice versa) of wells in the three-dimensional array that are both perpendicular to the z-axis would correspond to the x and y axes, respectively. In a preferred technique, a reagent is added into a plurality of inlets at two or more faces or surfaces of the three-dimensional array, in which the two or more surfaces of the array are adjacent to each other.

Typically, a device of the invention allows the addition of a reagent into the wells in a substantially parallel direction relative to an axis or plane of a plate of wells. If desired, the array wells can be designed or configured to have openings and/or conduits such that a nonperpendicular addition or removal or a fluid from one or more wells would be possible. Preferably, the device allows a flow of a reagent between wells mainly through the use of unobstructed openings between wells. When an elongated element is used, a reagent preferably flows while in contact with the elongated element. When used in an array, an elongated element preferably at least partially obstructs one or more openings in at least one of the wells. The elongated element includes, but is not limited to, a pin, rod, and tube.

As used herein, the term “pin” includes any suitable elongated element. A pin is preferably made of a relatively nonreactive material such as polypropylene, polyethylene, or teflon. A pin can also be made of metals or alloys such as stainless steel or aluminum. The pin's radial cross-section may have a substantially round, square, triangular, or other shapes. The pin may extend over two or more array wells.

A pin diameter or width is preferably chosen to optimize the flow of reagent or solvent for a given parameter or combination of parameters that include well opening dimension, well dimension, or number of wells in which the reagent or solvent are to be added. Preferably, the pin diameter is less than the diameter of a well opening so that a reagent or solvent can flow from one well to the next when a pin is positioned across a well. More preferably, the pin diameter and the diameter of the well opening are chosen such that a fluid can flow freely from one array well to another when the fluid height in a well sufficiently exceeds the height of a lower well opening (as measured from a bottom portion of the well).

When used as a sealing element, a pin may include O-rings to enhance or facilitate the sealing or isolation of the wells. Preferably, sealing is achieved when the sealing elements are pushed or pulled individually or simultaneously (e.g., by installing one or more bars that connect several sealing elements) such that the O-rings block the flow of the reagent when the O-rings lie within or coincide with the well wall. When the sealing elements are pushed or pulled such that the O-rings are not coincident with the well walls, one or more reagents or solvents may then be introduced into the wells. The O-rings are preferably made of materials such as perfluoroelastomers sold under the name “KALREZ” (sold by Dupont Dow Elastomers, Wilmington, Del.), PTFE, or other materials that are resistant or non-reactive to the reagents or solvents used during the synthesis.

The sealing elements may include channels such as holes or grooves for reagent or solvent delivery. As used herein, the term “channels” refers to openings, such as holes or grooves in the elongated elements, through which fluid can flow. Thus, an elongated element or sealing element with channels can be used as a fluid conduit or fluid introduction conduit. As used herein, a “fluid conduit” or “fluid introduction conduit” generally refers to means for introducing or adding fluid into one or more receiving elements such as the wells of an array. A fluid introduction conduit can be a tube, elongated needle, pin, rod, or cylinder. The fluid introduction conduit preferably has one or more channels. For example, a fluid introduction conduit can be a pin with multiple channels along its length. That pin can be inserted through a row of wells so that fluid can be separately added in each well. Alternatively, a fluid introduction conduit, such as a thin hollow tube, may have openings only on either end of the tube.

In one variation of a fluid introduction conduit, the distance between channels in a pin, for example, preferably coincides with the length of two opposite sides of a well. With this type of fluid introduction conduit, which can also function as a sealing element, substantial sealing is achieved when the conduits are pushed or pulled individually or simultaneously such that the well wall material covers the holes or grooves in the sealing elements and substantially no fluid leaks from the well through the holes in the sealing elements.

The conduits or sealing elements may be made of at least one type of material such as stainless steel, aluminum, PTFE, polypropylene, polyethylene, or a combination of materials such as polypropylene tubes that contains a stainless steel rod at the center. Preferably, the conduits or sealing elements are in the form of stainless steel tapered pins.

FIG. 11A shows a configuration of a plate 110 comprising an array of reactor wells 112 containing pins 114 and solid-phase support 116. As shown in FIG. 11A, the pins 114 may extend over at least two wells 112, each well having at least two openings 118 through which an elongated element 114 may be inserted. In FIG. 11A, the plate 110 includes openings 118 on at least two opposite sides 130, 132 of the plate 110. Elongated elements 114, such as pins, may be inserted or withdrawn from either side 130, 132 of the plate 110. FIG. 11B also shows a three-dimensional multiparallel synthesizer 118 with connections 120, 122, such as tubes, to reagent/solvent reservoirs 124, 126. The synthesizer 118 comprises plates with inlet ports 140, in which sealing between plates is achieved using two clamping plates 134, 136 which are clamped tight using, for example, screws 138. The right side of the synthesizer 118 shows openings 142 through which fluid may be introduced into the wells of the synthesizer 118.

Different types of linkers may be used in the various aspects of the invention. A linker covalently attaches molecules to the solid-phase support. The choice of a particular linker depends on factors such as the particular product or intermediate to be synthesized and the stability of a linker. Different types of linkers are known in the art and they are preferably attached to the solid-phase supports using standard solid-phase chemistry techniques. Preferably, the linkers are those based or adapted from protecting group chemistry.

Linkers that can be used with an array or method of the invention include acid labile linkers, nucleophile labile linkers, safety-catch linkers, traceless linkers, fluoride labile linkers, and photo-labile linkers. An advantage of nucleophile labile linkers is that it can be used to introduce a moiety or functional group during the cleavage step. Safety-catch linkers allows cleavage of an activated linker using mild conditions. Photo-labile linkers can be used under mild conditions and the process can be selective.

Other examples of linkers that may be used with an array or method of the invention include, but are not limited to, rink amide linkers, hydroxymethylphenoxy linker (HMP linker), backbone amide linker, trityl alcohol linker, disulfide linker, sulfoester linker, benzylhydryl or benzylamide linker, ortho-nitrobenzyl-based linker, nitroveratryoxycarbonyl-based linkers, and phenacyl based linkers.

The following examples illustrate the use of some of the above linkers. Rink amide linkers, which are commercially available, can be used with activated carboxylic acids which cleave to form primary carboxyamides. Rink amide linkers can also be loaded with sulfonyl chorided to produce primary sulfonamides. When using rink amide linker, cleavage is normally performed using 20% TFA/DCM. Solid-phase supports with HMP linker can be used to link carboxylic acids, phenols, and amines. In this case, cleavage via acidolysis produces the original functional group. The carboxylic acids can be coupled using N,N′-diisopropylcarbodiimide (DIC)/N,N-dimethylaminopyridine through imidate or Mitsunobu chemistry. With HMP linker, cleavage is normally performed with about 20% or higher concentrations of trifluoroacetic acid (TFA)/dichloromethane (DCM). Trityl alcohol linker can be used to link carboxylic acids, alcohols, phenols, and amines. Cleavage via acidolysis produces the original functional group. Hyperlabile linker links phenols, amines, and carboxylic acids. Cleavage by acidolysis recovers the original functional group.

Cleavage of the synthesis products can be performed using techniques known in the art. In one aspect, a product is cleaved from the solid-phase support via an intramolecular reaction that removes the compound from the solid-phase support without leaving any trace of the site of attachment. See DeWitt, et al., Proc. Natl. Acad. Sci. U.S.A., 1993, 90: 6909-6913, which is incorporated herein in its entirety.

Following the cleavage step, the cleaved compounds may be analyzed or characterized using one or more analytical techniques such as mass spectrometry, liquid chromatography, NMR, or a combination of techniques such as LC-UV/MS. On-site or on-bead analysis may be performed using techniques such as magic angle spinning NMR or FT-IR spectrometry. The applications of one or more of these techniques in solid-phase synthesis have been described in several publications including, for example, Chu et al., J. Org. Chem., 58: 648-652 (1993); Fitch et al., J. Org. Chem., 59: 7955-7956 (1994); Gao et al., J. Med. Chem., 39: 1949-1955 (1996); P. A. Keifer, J. Org. Chem., 61: 1558-1559 (1996); Metzger et al., Angew. Chem. Int. Ed., 32: 894-896 (1993); Stevanovich and Jung, Anal. Biochem., 212: 212-220 (1993); and Youngquist et al., Rapid Commun. Mass Spectrom., 8: 77-81 (1994), all of which are incorporated herein in their entirety.

Various types of solid-phase chemistries for the combinatorial synthesis of non-oligomeric small molecules are well-known in the art, and they may be used in conjunction with the various aspects of the invention. Examples of these solid-phase techniques include, but are not limited to, those described in Gordon et al., J. Med. Chem., 37: 1385 (1994), Patel and Gordon, Drug Disc. Today, 4: 134-144 (1996); Fruchtel and Jung, Agnew. Chem., 35: 17-41 (1996); Thompson and Ellman, Chem. Rev., 96: 555-600 (1996); Bunin and Ellman, J. Am. Chem. Soc., 114: 10997 (1992); Gallop et al., U.S. Pat. No. 5,525,734, Bunin et al., Proc. Natl. Acad. Sci. U.S.A., 91: 4708 (1994); Plunkett and Ellman, J. Am. Chem. Soc., 117: 3306 (1995); Hobbs DeWitt et al, Proc. Natl. Acad. Sci. U.S.A., 90: 6909 (1993); Murphy et al., J. Am. Chem. Soc., 117: 7029 (1995); Holmes et al, J. Org. Chem., 60: 7328 (1995); Holmes, U.S. Pat. No. 5,549,974; Gordon and Steel, Bioorg. Med. Chem. Lett., 5: 47 (1995); Patek et al, Tetrahedron Lett. 36: 2227 (1995); Szardenings et al, Tetrahedron, 53: 6573 (1997); Beebe et al., J. Am. Chem. Soc., 114: 10061 (1992); Moon et al, J. Org. Chem., 57: 6088 (1992); Pei and Moos, Tetrahedron Lett., 35: 5825 (1994); and and Maclean, Proc. Natl. Acad. Sci. U.S.A., 94: 2805 (1997), each of which is incorporated herein in its entirety.

In one aspect of the invention, the fluids include small molecule reactants. The chemistry of several non-peptide libraries have been described in the art. See, for example, Cho et al., Science, 261: 1303-1305; DeWitt et al., Proc. Natl. Acad. Sci. U.S.A., 90: 6909-6913; Simon et al, Proc. Natl. Acad. Sci. U.S.A., 89: 9367-9371; Zuckermann et al., J. Amer. Chem. Soc., 114: 10646-10647; and Zuckermann et al., J. Med. Chem., 37: 2678-2685, all of which are incorporated herein in their entirety. The chemistry and methods described in these references may be used in conjunction with the devices and methods of the invention. In another aspect, the fluid includes one or more biomolecules.

The present invention includes a method for synthesizing or screening an array of compounds. Preferably, the method comprises introducing a fluid into a plurality of wells through at least one opening in a wall between at least two of the plurality of wells. In one aspect, the method includes introducing a fluid into several sets of wells, in which at least three of the sets of wells are oriented perpendicularly with respect to each other. The fluid may be added into a plurality of inlets at two or more adjacent faces or surfaces of the three-dimensional array. An elongated element that extends between at least two sides of a well may be used when performing an array operation such as reagent addition or removal.

FIG. 2 shows a synthesis protocol using a 2×2×2 array as a representative example. The process begins with the removal of four pins from one side of the cube. The additions of the two components A₁ and A₂ to the first two layers of the array are carried out simultaneously. The pins inserted in a perpendicular direction provide the necessary sealing and avoid the undesired mixing between reactants A₁ and A₂. Following the washing cycle, the process is repeated using the next set of reagents B₁ and B₂ by changing the direction of reagent addition by 90°. Following another washing cycle, the four pins are reinserted as shown in FIG. 2B. This operation allows the last two components C₁ and C₂ to be delivered on an adjacent face or surface of the array, thus completing the synthesis process. At the end of the synthesis, the layers of the array can be physically separated to allow the removal of the material from the reactor wells either prior to or following the solid-phase cleavage step. Although compact in size, the device of the invention allows the preparation of milligram quantities of final products, which is sufficient for performing hundreds of biological assays.

Extension of the method above to a 5×5×5 array is shown in FIG. 3. This array is capable of delivering 125 spatially localized and positionally encoded components. This array allows coupling steps to be conducted within a given layer of the array without cross-contamination. Teflon gaskets may be used to achieve fluid-tight seals between the layers. Other types of gaskets, such as those made of aluminum, or aluminum coated with a polypropylene film or other types of polymeric film, may also be used. Each well is equipped with a single 12.5 mm SynPhase™ lantern. Based on the typical loading capacity of 35 μmol/lantern, the synthesis can produce 5-15 mg of individual library members. Each well has four openings allowing for insertion of tapered stainless steel pins of the appropriate diameter to achieve fluid-tight sealing when the pin is inserted to block the appropriate openings. Removal of the pin connects the adjacent reactors, thus allowing the addition of reagents.

A method according to the present invention may include introducing a fluid into wells of the array, and inserting a plurality of removable elongated elements each of which is positioned across or spans at least two sides of a well when in a sealing position. In another aspect, the method for synthesizing or screening an array of compounds includes adding a fluid in a parallel direction relative to an axis or plane of a plate. A method may also include adding a first fluid or first solid to a first set of wells in a parallel direction relative to an axis or plane or a plate, and adding a fluid into a second set of wells. Further addition steps can be performed, including other types of array operations such as reagent removal or well or solid-phase support washing.

A method for synthesizing or screening an array of compounds may also include adding a first fluid or first solid to a first two layers of a device comprising wells, inserting a plurality of elongated elements into the wells following the addition of a first fluid or first solid to the first two layers of the device comprising wells, and adding a second fluid or second solid in a direction perpendicular to a direction of addition of the first fluid or first solid, wherein points of entry of the fluid through the inlets define a line of direction of addition.

In one aspect, the method of the invention includes performing at least one washing step after the addition of the fluids or solids. Preferably, a washing step is performed after each addition of a fluid using suitable solvents such as water, organic solvents, aqueous or organic solutions, or combinations of suitable solvents.

Preferably, the addition of the fluid is performed at least three times. The fluid may be added using delivery systems or devices such as syringes or tubes. Preferably, the fluids are added with the aid of a pump. Different types of pumps such as syringe pumps, peristaltic pumps, piston metering pumps, and diaphragm pumps are commercially available. Preferably, the pump is a syringe pump. The pumps suitable for use in the various aspects of the invention are those having components that are preferably resistant to the solvent or reagents used in the synthesis. Preferably, the pumps can be operated under a wide range of solution temperatures.

Inert gas such as argon or nitrogen may be used to allow pressurized delivery or transfer of a reagent. For example, a pressurized inert gas such as argon or nitrogen may be applied to a tube to force a fluid from a reservoir into the array. The reservoirs themselves may be subjected to pressure to allow or facilitate the transfer of a reagent or solvent from a reservoir into the wells of the array. In one aspect, one or more valves, which are electronically controlled, such as solenoid valves with a plurality of ports, are used in the reagent or solvent transfer.

### EXAMPLES

The following examples are provided by way of illustration and should not be viewed as limiting the scope of the present invention.

### Example 1

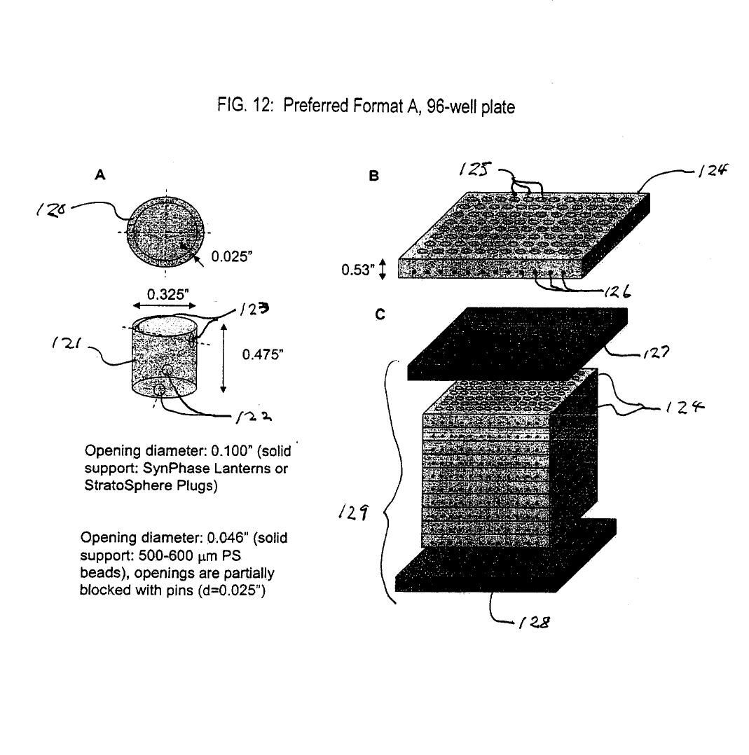

FIG. 12 shows a preferred 96-well array configuration 129 according to the present invention. Sealing between the plates 124 with openings 126 can be achieved by using, for example, a top clamping plate 127 and a bottom clamping plate 128. Screws may be used to clamp tight the plates 124 using the clamping plates 127, 128. FIG. 12 also shows a top view 120 and a side view 121 of a representative well with top openings 123 and bottom openings 122. The average diameter of the openings 122, 123, 126 for this array format 129 is preferably between about 2.3 mm to about 2.7 mm, more preferably about 2.5 mm when a solid-phase support such as a SynPhase lantern or StratoSphere plug is used. The solid-phase support, such as a SynPhase lantern or StratoSphere plug, can be subdivided into several fragments. When polystyrene beads having diameters ranging from about 0.5 mm to about 0.6 mm are used as solid-phase support, the average diameter of the openings is preferably about 1.2 mm. If desired, one or more openings 122, 123, 126 can be partially blocked using an elongated element such as a pin, rod, or tube having a diameter of about 0.7 mm. Preferably, a reagent is introduced into the wells 125 of a delivery device such as a syringe or syringe array. The reagents may also be added into the wells 125 through the used of pressurized reagent reservoirs. Interconnected tubes that are connected to reagent reservoirs may be inserted into the inlet ports of the array for simultaneous addition of one or more reagents. Other methods of adding reagents may also be used.

### Example 2

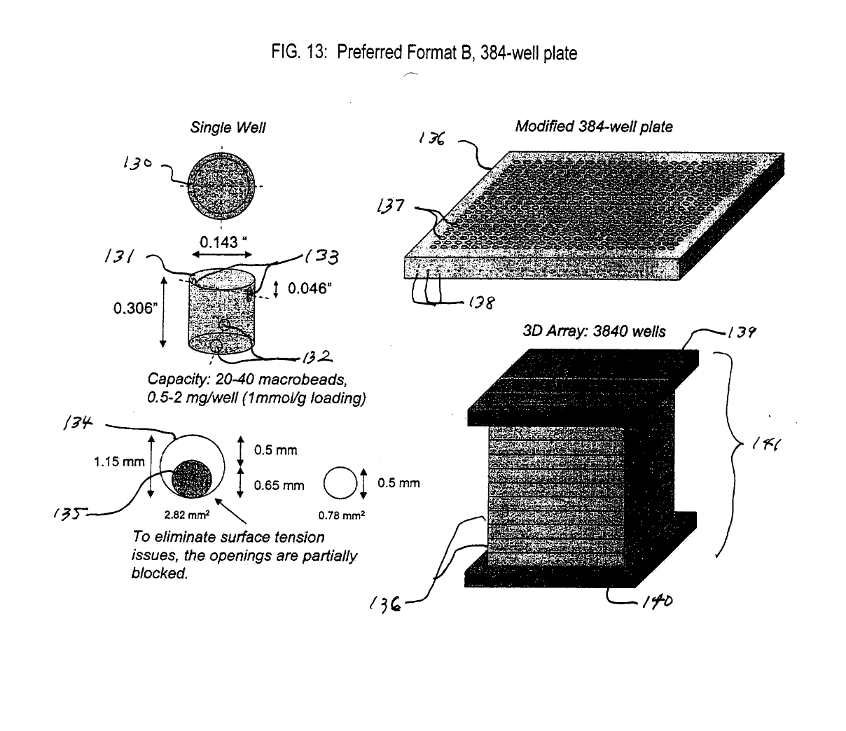

An array 141 based on 384-well plates 136 is a highly preferred array format. FIG. 13 shows a 384-well plate array configuration 141 according to the present invention. As in FIG. 12, sealing between the plates 136 can be provided by using a top clamping plate 139 and a bottom clamping plate 140. FIG. 13 also depicts a top view 130 and a side view 131 of a representative well of a plate 136. The side view 131 of the well shows a pair of top openings 133 and a pair of bottom openings 132. The diameters of openings 132, 133, 138 in a well 137 in a 384-well plate 136 is preferably between about 0.5 mm to about 3 mm, more preferably between about 0.75 mm to about 1.5 mm, most preferably between about 1.0 mm to about 1.3 mm. For a well 136 illustrated in FIG. 13, the preferred dimensions are about 3.6 mm in diameter, about 7.8 mm in height or depth, with at least one opening having an average diameter of about 1.2 mm. FIG. 13 shows the relative diameters of a well opening 134 and an elongated element 135. With these dimensions, each well would contain, for example, between about 20-40 macrobeads with a corresponding loading of about 1 mmol/g. An average diameter of an opening of about 1.2 mm, for example, allows the addition and withdrawal of reagents to be carried out without significant complications arising from surface tension effects. When polystyrene beads with diameters ranging from between about 500-600 μm are used as solid-phase support, the average diameter of the openings is preferably between about 1.0 mm to about 1.3 mm. A reagent can be introduced into the wells through the use of a delivery device such as a syringe or syringe array. The reagents may also be introduced into the wells through the used of pressurized reagent reservoirs. Interconnected tubes that are connected to reagent reservoirs may be inserted into the inlet ports of the array for simultaneous addition of one or more reagents. Other methods of adding a reagent may be used.

### Example 3

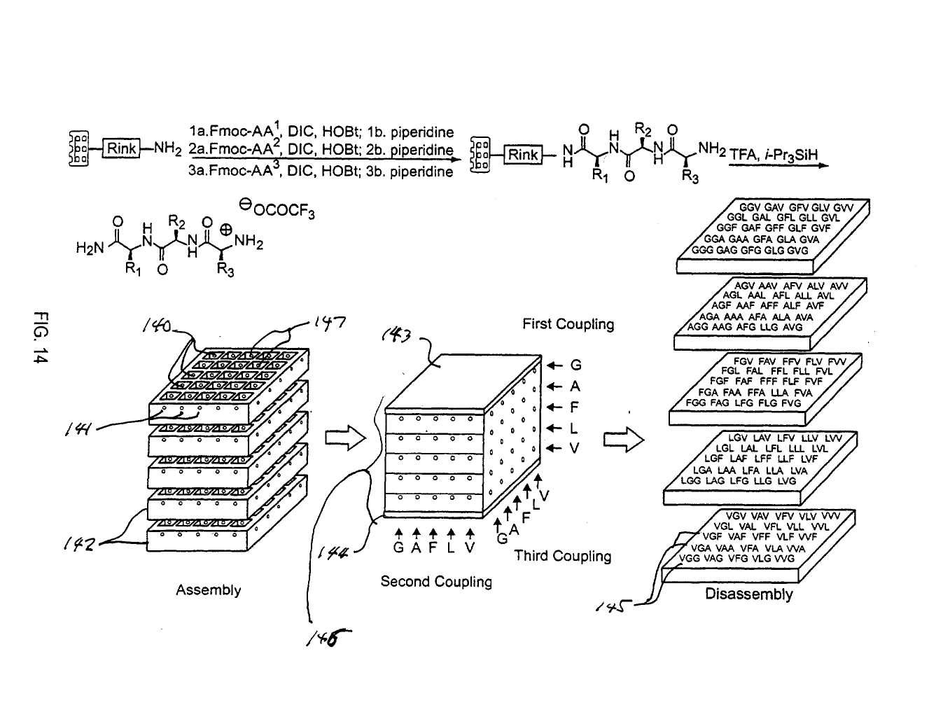

A synthesis of a 125-peptide library using the system shown in FIG. 14. FIG. 14 shows a three-dimensional array 146 that includes a top layer or plate 143 and a bottom plate 144. The top and bottom plates 143, 144 were used to clamp tight the plates 142 in-between the top and bottom plates 143, 144 to afford sealing between the plates 142. As shown in FIG. 14, the plates 142 have openings 141, 147 in the side walls of the wells 140 and also on the outer sides of the plates 142, in which elongated elements such as pins may be inserted through these openings 141, 147. The library was generated by randomization of the tripeptides 145 at each position with five amino acids Gly, Ala, Phe, Val, and Leu. A conventional 9-fluorenylmethoxycarbonyl (Fmoc)-solid-phase peptide synthesis using N-hydroxybenzotriazole (monohydrate) (HOBt)/DIC coupling protocol using commercially available SynPhase D-series PA lanterns with Rink linker. The three-dimensional arraying of the five amino acids is schematically illustrated in FIG. 14. The synthesis was carried out within the expected 10-hour period. Examination of 25 peptides 145, randomly selected from the final array using ¹H-NMR spectroscopy and HPLC-MS confirmed their identities and high chemical purity. This particular example of parallel synthesis can be extended to the preparation of a 1,000-compound (or greater) three-dimensional arrays equipped with readily available solid-phase supports such as 500-600 μm polystyrene beads.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

1. A device for multiparallel synthesis or screening of a chemical library comprising:
a plate comprising a two-dimensional array of wells for receiving fluids, each well comprising a bottom and side walls; and
openings in the side walls between at least some of the wells, through which the fluids can flow between adjacent wells; and
at least one inlet for the plate whereby fluids can be introduced into the array of wells in the plate and flow between adjacent wells through the openings in the side walls.

2. The device of claim 1 comprising at least a first inlet and a second inlet for the plate and wherein the openings in the side walls are arranged so that fluid introduced through the first inlet flows into a first group of wells and fluid introduced through the second inlet flows into a second group of wells, but the fluid introduced through the first inlet does not flow into the second group of wells and fluid introduced through the second inlet does not flow into the first group of wells.

3. The device of claim 2 further comprising a fluid supply associated with each of the first inlet and second inlet, and a fluid supply controller whereby the same or different fluids are selectively added to different groups of wells.

4. The device of claim 1 wherein the two-dimensional array comprises rows of wells, and wherein the openings are arranged so that fluid flows between adjacent wells in each of the rows of wells, but not between the rows of wells.

5. The device of claim 4 further comprising an inlet associated with each row, a fluid supply associated with each inlet, and a fluid supply controller, whereby the same or different fluids are selectively added to different rows of wells.

6. The device of claim 5, wherein a first plate and a second plate are configured and aligned so that rows of wells in the first plate are parallel to rows of wells in the second plate and wherein the fluid supply controller is adapted to simultaneously add a common fluid to multiple rows in the first plate or the second plate under a first setting, and alternatively to simultaneously add a common fluid to multiple rows of the first plate and the second plate under a second setting.

7. The device of claim 6 wherein, under the first setting, the common fluid is added to all of the rows in the first plate or the second plate, and, under the second setting, the common fluid is added to all of the rows in a plane that encompasses the first plate and the second plate.

8. The device of claim 1, wherein the array is in fluid communication with at least one other array.

9. The device of claim 1, wherein the wells are made of at least one material selected from a group consisting of polymers, resins, metals, glass beads, silica supports, gel or gel-type solid-phase supports, encapsulated gel solid-phase supports, macroporous supports, modified surfaces, and composite particles.

10. The device of claim 1, wherein the smallest side of each of the wells ranges between about 3.4 mm to about 3.8 mm.

11. The device of claim 1, wherein each of the wells has a depth that ranges between about 6 mm to about 9 mm.

12. The device of claim 1 further comprising a solid-phase support located in at least some of the wells.

13. The device of claim 1, wherein at least about one milligram of a reaction product is produced from a synthesis reaction in at least some of the wells.

14. The device of claim 1, wherein the wells comprise at least one type of solid-phase support that contains preformed functionalities or linkers.

15. The device of claim 1, wherein a functionalization, linking, or cleavage step is performed in at least some of the wells.

16. The device of claim 1, wherein at least one of the fluids comprises small molecule reactants.

17. The device of claim 1, wherein at least one of the fluids comprises molecules with a dendritic structure.

18. A device for multiparallel synthesis or screening comprising:
an array comprising rows of wells for receiving fluids, each well comprising a bottom and side walls;
openings in the side walls, the openings being aligned along each of the rows of wells; and
at least one elongated element adapted to be inserted through at least some of the openings along at least one of the rows of wells;
wherein a flow of fluids along the at least one of the rows of wells is controlled by the elongated element's reducing or closing the openings it passes through.

19. The device of claim 18, wherein the elongated element is operated by selectively being inserted into and removed from the row of wells.

20. The device of claim 18, wherein the elongated element is operated by changing an extent of insertion less than the length of a single well.

21. The device of claim 18, wherein the elongated element is operated by rotating the elongated element about its longitudinal axis.

22. The device of claim 18, wherein the elongated element comprises a channel running therethrough, through which a fluid can be added to the wells of a row.

23. The device of claim 18, wherein the elongated element narrows at least one of the openings to a sufficient degree to preclude fluid flow therethrough.

24. The device of claim 18, wherein the elongated element forms a complete seal of the openings.

25. The device of claim 18, wherein the elongated element is selected from a group consisting of a pin, needle, rod, and tube.

26. A device for multiparallel synthesis or screening comprising:
an array comprising wells for receiving fluids, the wells being arranged in rows and columns, each of the wells comprising a bottom and side walls;
a first set of openings in the side walls, the openings in the first set of openings being aligned along each of the rows;
a second set of openings in the side walls, the openings in the second set of openings being aligned along each of the columns;
a first set of elongated elements, each adapted to be inserted through the first set of openings in a single row; and
a second set of elongated elements, each adapted to be inserted through the second set of openings in a single column;
wherein a flow of fluids along each of the rows is controlled by one of the first set of elongated elements passing therethrough by reducing or closing one or more openings along each of the rows; and wherein the flow of fluids along each of the columns is controlled by one of the second set of elongated elements passing therethrough by reducing or closing one or more openings along each of the columns.

27. A device for multiparallel synthesis or screening comprising:
(a) a three-dimensional array formed from at least two plates comprising a plurality of wells for receiving a fluid; and
(b) means for introducing the fluid into a plurality of inlets at two or more adjacent surfaces of the three-dimensional array,
wherein the fluid flows from a first of the plurality of wells to one or more other wells selected from the plurality of wells through openings in the plurality of wells, and a bottom portion of each of the plurality of wells is sealed to prevent the flow of the fluid from a well of a first plate to a well of a second plate.

28. A device for multiparallel synthesis or screening comprising:
(a) an array formed from at least two plates comprising a plurality of wells for receiving a fluid; and
(b) means for allowing a flow of the fluid between at least two of the plurality of wells in a substantially parallel direction relative to a plane of one of the at least two plates,
wherein the fluid is introduced through an opening on one side of the array, and the fluid flows from a first of the plurality of wells to one or more other wells selected from the plurality of wells through openings in one or more of the plurality of wells.

29. A device for multiparallel synthesis or screening comprising:
an array formed from at least two plates, each comprising a plurality of wells for receiving a fluid; and
means for introducing a fluid into at least three sets of wells selected from the plurality of wells,
wherein each set of wells is oriented perpendicularly relative to two of the three sets of wells, the fluid is introduced through an opening on one side of the array, and the fluid flows from a first of the plurality of wells to one or more other wells selected from the plurality of wells through openings in at least one of the plurality of wells.

30. A device for multiparallel synthesis or screening comprising:
(a) an array formed from at least two plates comprising a plurality of wells for receiving a fluid;
(b) means for introducing the fluid into one or more of the plurality of wells; and
(c) a plurality of removable sealing elements each of which extends between at least two openings in at least one of the plurality of wells when in a sealing position.

31. The device of claim 30, wherein at least one of the plurality of removable sealing elements is selected from a group consisting of a rod-shaped sealing element, tapered sealing element, and a cylindrical sealing element.

32. The device of claim 30, wherein the sealing elements are stainless steel tapered pins.

33. The device of claim 30, wherein the fluid is introduced through one or more channels in at least one of the plurality of sealing elements.

34. A device for multiparallel synthesis or screening of a chemical library comprising:
a three-dimensional array formed from at least two plates joined together, each plate comprising a two-dimensional array of wells for receiving fluids, the wells in each plate being formed into rows and columns, each well comprising at least a bottom and four side walls;
openings in all four of the side walls of all of the wells in the arrays, a first set of the openings allowing fluids to flow between adjacent wells along at least one of the rows, and a second set of openings allowing fluids to flow between adjacent wells along at least one of the columns; and
a first set of inlets for introducing fluids into each of the rows; and
a second set of inlets for introducing fluids into each of the columns;
wherein the first set of openings is adjacent the bottom of each of the wells and the second set of openings is adjacent an upper portion of each of the wells, and wherein the level of fluid in the wells is maintained below the second set of openings when the device is in a first orientation with the bottom of each of the wells below the upper portion of each of the wells, and the level of fluid in the wells is maintained below the first set of openings when the device is inverted to a second orientation with the upper portion of each of the wells below the bottom of each of the wells; thereby selectively allowing fluid communication between the wells in each of the rows but not between the wells in each of the columns when the device is in its first orientation, and allowing fluid communication between the wells in each of the columns but not between the wells in each of the rows when the device is inverted to its second orientation.

35. The device of claim 34 further comprising a solid-phase support in at least some of the wells.

36. The device of claim 34 further comprising a fluid introduction conduit associated with at least some of the inlets.

37. The device of claim 36 wherein a diameter of the fluid introduction conduit is smaller than a diameter of at least one of the openings.

38. The device of claim 37 wherein the fluid introduction conduit passes through at least one of the inlets and through the openings in a row whereby fluid is first introduced into a last well in the row and the fluid flows into the remaining wells in the row through the openings.

39. The device of claim 37 wherein the fluid introduction conduit is selectively inserted into and retracted from the row.

40. A device for multiparallel synthesis or screening comprising:
a three-dimensional array comprising at least two plates, comprising wells for receiving fluids arranged in rows and columns, each well comprising a bottom and side walls;
a first set of openings in the side walls, the openings in the first set of openings being aligned along each of the rows;
a second set of openings in the side walls, the openings in the second set of openings being aligned along each of the columns;
a first set of elongated elements, each adapted to be inserted through the first set of openings; and
a second set of elongated elements, each adapted to be inserted through the second set of openings;
wherein the flow of fluids along each of the rows is controlled by an elongated element of the first set passing therethrough by reducing or closing the openings therein; and wherein the flow of fluids along each of the columns is controlled by an elongated element of the second set passing therethrough by reducing or closing the openings therein.

41. A method for synthesizing or screening compounds comprising:
introducing a first fluid into a plurality of wells of an array formed from at least two plates through at least one opening in each wall of the plurality of wells; and
introducing a second fluid into the plurality of wells through the at least one opening in each wall of the plurality of wells,
wherein a first and second fluid is introduced through an opening on one side of the array, and the first and second fluid flow from a first of the plurality of wells to one or more other wells selected from the plurality of wells in a substantially parallel direction relative to a plane of one of the at least two plates.

42. The method according to claim 41 further comprising inserting at least one elongated element into at least one opening in one or more of the plurality of wells.

43. The method according to claim 42, wherein the at least one elongated element extends between at least two openings in at least one of the plurality of wells when in a sealing position.

44. The method according to claim 41 further comprising performing one or more washing steps after introducing one or more fluids into the plurality of wells.

45. The method according to claim 41, wherein the fluid is added using a fluid introduction conduit.

46. The method according to claim 41, wherein between about 0.01 milligram to about 20 milligram of a reaction product is produced from a mixing of at least two fluids.

47. The method according to claim 41, wherein the fluid is added using a pump.

48. The method according to claim 41 further comprising using at least one type of solid-phase support made of at least one material selected from a group consisting of polymers, resins, metals, glass beads, silica supports, gel or gel-type solid-phase supports, encapsulated gel solid-phase supports, macroporous supports, modified surfaces, and composite particles.

49. The method according to claim 41, wherein at least one step selected from a group consisting of a functionalization step, linking step, and a cleaving step is performed in at least one of the plurality of wells.

50. The method according to claim 41, wherein the fluid comprises small molecule reactants.

51. The method according to claim 41, wherein the fluid comprises molecules with a dendritic structure.

52. A method for synthesizing or screening a library of compounds comprising using the device of claims 1, 18, 26, 27, 28, 29, 30, 34, or 40 to produce between about 0.01 milligram to about 20 milligram of at least one member of the library of compounds.

53. A method for synthesizing or screening compounds comprising:
introducing a fluid into a first set of wells of an array;
introducing a fluid into a second set of wells of the array that lie perpendicularly to the first set of wells of the array; and
introducing a fluid into a third set of wells of the array that lie perpendicularly to each of the first set of wells and the second set of wells of the array.

54. The method of claim 53, wherein any of the first set of wells, second set of wells, and the third set of wells corresponds to a column of wells or a row of wells.

55. A method for synthesizing or screening compounds comprising:
introducing a fluid into a first set of inlets at a first surface of a three-dimensional array that comprises a plurality of wells; and
introducing a fluid into a second set of inlets at a second surface of the three-dimensional array,
wherein the first surface of the three-dimensional array lies adjacent to the second surface of the three-dimensional array.

56. A method for synthesizing or analyzing compounds comprising:
introducing a fluid through a first set of openings in a first set of side walls in an array;
rotating the array by an angle; and
introducing a fluid through a second set of openings in a second set of side walls in the array.
